(19) United States
(12) Patent Application Publication
(10) Pub. No.: US 2020/0306076 A1
(43) Pub. Date: Oct. 1, 2020

(54) Anti-Snoring Systems

(71) Applicant: MARK CUZZETTO, BURNABY (CA)

(72) Inventor: MARK CUZZETTO, BURNABY (CA)

(21) Appl. No.: 16/900,191

(22) Filed: Jun. 12, 2020

Related U.S. Application Data
(63) Continuation-in-part of application No. 15/068,859, filed on Mar. 14, 2016, now abandoned.

(51) Int. Cl. A61F 5/56; A47G 9/10

(52) U.S. Cl. CPC A61F 5/56; A47G 2009/1018; A47G 9/10

(57) ABSTRACT

The anti-snoring assembly includes an anti-snoring body, with the anti-snoring body, including a top side, a triangle side 1.0, a triangle side 2.0, and a bottom side. The top side 2.0 includes a left surface and a right surface, and the triangle side 1.0 includes a triangle side 1.0 surface. The triangle side 2.0 includes a triangle side 2.0 surface, where the bottom side 2.0 includes a bottom side surface. The anti-snoring system is structured and arranged to be placed beneath a pillow of a user to provide the user with a favorable downward head angle for reducing or preventing snoring during sleep. Embodiments include arrangements where the triangle side 1.0 is flat, and both the triangle side 2.0 and the bottom side are triangular. Additionally, the left surface and the right surface of the top side of the anti-snoring system may each resemble a flat plane.

### RELATED APPLICATIONS

This application is a Continuation-in-Part of application Ser. No. 15/068,859, filed on Mar. 14, 2016, pending. The entire disclosure of application Ser. No. 15/068,859 is with this incorporated by reference.

### BACKGROUND OF THE INVENTION

The following includes information that may be useful in understanding the present invention(s). It is not an admission that any of the information provided herein is prior art, or material, to the presently described or claimed inventions, or that any publication or document that is explicitly or implicitly referenced is prior art.

### FIELD OF THE INVENTION

The present invention generally relates to the field of bedding materials and, more specifically, relates to anti-snoring systems.

### DESCRIPTION OF RELATED ART

Snoring is a condition that occurs during sleep and has been known to cause sleep deprivation to individuals that snore and those around them. Additionally, snoring may cause drowsiness, irritability, and lack of concentration during times of non-sleep. Sometimes snoring may be considered a minor affliction; however, individuals who snore may sometimes suffer from severe impairment of their lifestyle. Recent medical studies have associated snoring with the development of carotid artery atherosclerosis.

Specific treatments and methods of reducing chances or instances of snoring can be found, which may include waking the snoring individual. However, waking a snoring individual may cause sleep deprivation, which may aggravate or increase instances of snoring. Some devices may be available to attempt to reduce the chances of snoring; however, many of these devices are costly, complicated, and often ineffective. Therefore, an inexpensive and simple to operate solution is desired.

Several attempts have been made to solve the problems mentioned above, such as those found in U.S. and Foreign Pat. and Pub. Nos. U.S. Pat. No. 4,848,360 to Palsgard et al., US 2014/0069777 to Derner, U.S. Pat. No. 7,469,434 to Drucker, U.S. Pat. No. 7,100,227 to Frisbee, U.S. Pat. No. 6,386,201 to Fard, U.S. Pat. No. 7,594,288 to Holliday et al., US 2012/0079660 to Chen, US 2012/0060846 to Leoniak et al., US 2014/0296747 to Herrnsdorf, US 2015/0265075 to Liu et al., CN 204,192,189 to Liang et al., CN 202,619,948 to Qi et al., CN 201,085,722 to Jianguo, CN 2,235,792 to Yong, EP 0765647 to Berends. DE 19,930,818 to Golriz, CA 2,478,289 to Koops, WO 01/32112 to Botha, and EP 1,488,766 to Screen.

U.S. Pat. No. 6,671,907 Zuberi describes a sleep apnea avoidance process that includes selecting a pillow having a pair of sides, angled at a predetermined angle, and shaped to hold a person's head face down on one side. The selected pillow has a pair of arm openings thereunder to position a person's arm to assist in holding a person's head face down on the pillow angle side such that the user can use one or the other arms when placing the head on one or the other angled side of the pillow. The process includes resting on one of the pillow's angled sides with one arm placed through the arm opening.

This art is representative of bedding accessories. However, none of the above inventions and patents, taken either singly or in combination, is seen to describe the invention as claimed.

### BRIEF SUMMARY OF THE INVENTION

Because of the foregoing disadvantages inherent in the known bedding materials art, the present invention provides a novel anti-snoring system. The present invention is to provide a device to be used in conjunction with a pillow to provide a user with a head position during sleeping that reduces or eliminates snoring.

An anti-snoring system comprising a body is disclosed. The body comprises a top side with a front surface that faces upward from a bed. The top side has a left surface and a right surface meeting the left surface, a triangle side 1.0 comprising a back surface, a triangle side 2.0 comprising a top surface, and a bottom side comprising a bottom surface. The anti-snoring system is structured and arranged to support a pillow to provide a user with a downward head angle useful for reducing and preventing snoring during sleep.

In some embodiments, the triangle side 1.0 is planar, the triangle side 2.0 and the bottom side are triangular, or the left surface and the right surface each comprise a flat plane.

In some embodiments, the left and right surfaces are rectangular. In some embodiments, the left and right surfaces of the top side each comprise a curvilinear plane. In some embodiments, the left and right surfaces of the top side of the body are semicircular.

The body can have a washable material or a flame-retardant material. In some embodiments, an organic or non-organic closed-cell foam material.

In some embodiments, the body does not contain armholes. In some embodiments, the body is 2 inches tall. In some embodiments, the system further comprises a kit including the anti-snoring assembly, a pillow, a pillowcase, and a set of user instructions.

In some embodiments, the anti-snoring system described in this document does not have armholes. In some embodiments, the anti-snoring system places a user's head at 0 to 25°, 0 to 20°, 0 to 15°, 5 to 25°, 5 to 20°, 5 to 15°, 10 to 20°, or 15°. In some embodiments, the anti-snoring system comprises a foam cushion and, in some embodiments, does not contain rigid arcuate components.

These embodiments do not represent sleep treatments. Sleep apnea and snoring are two completely different problems, requiring different solutions. While in some cases treating sleep apnea and snoring may seem similar, not all methods of treating or preventing snoring are suitable for treating sleep apnea and vice versa.

This document also discloses a method of using an anti-snoring system. The method comprises the steps of providing an anti-snoring system, providing a pillow and pillowcase, removing the pillow from the pillowcase, placing the pillow on top of the anti-snoring system, inserting the anti-snoring system and the pillow into the pillowcase, placing the anti-snoring system, the pillow and the pillowcase upon a bed, and placing a head of a user upon the anti-snoring system, the pillow, and the pillowcase. The method may further include the optional steps of removing the head of the user from the anti-snoring system, the pillow, and the pillowcase and removing the anti-snoring system from the pillowcase.

The present invention holds significant improvements and serves as an anti-snoring system. For purposes of summarizing the invention, certain aspects, advantages, and novel features of the invention have been described herein. It is to be understood that not necessarily all such advantages may be achieved following any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention, anti-snoring systems, constructed and operative according to the teachings of the present invention.

FIG. 1 shows a perspective view illustrating an anti-snoring system during an ‘in-use’ condition.

FIG. 2 is a perspective view illustrating an anti-snoring assembly.

FIG. 3 is an end view illustrating a pillow placed upon the anti-snoring assembly.

FIG. 4 is a perspective view illustrating an anti-snoring assembly.

FIG. 5 is a flowchart illustrating a method of use for the anti-snoring system.

FIG. 6A is a perspective view illustrating an anti-snoring assembly.

FIG. 6B is a perspective view illustrating an anti-snoring assembly.

FIG. 7 is a perspective view illustrating an anti-snoring assembly.

FIG. 8 is an end view illustrating an anti-snoring assembly.

FIG. 9 is a perspective view illustrating an anti-snoring assembly.

### DETAILED DESCRIPTION

As discussed above, embodiments of the present invention relate to a bedding material and, more particularly, to an anti-snoring system used to prevent or to improve the number of instances of snoring during sleep.

Studies have shown that an important cause for snoring is the relaxing of the jaw. The jaw draws closer to the back of the neck, which then restricts air flowing to and from the lungs. In some embodiments, the device pushes the jaw away from the neck, allowing for unobstructed breathing and reducing or eliminating snoring.

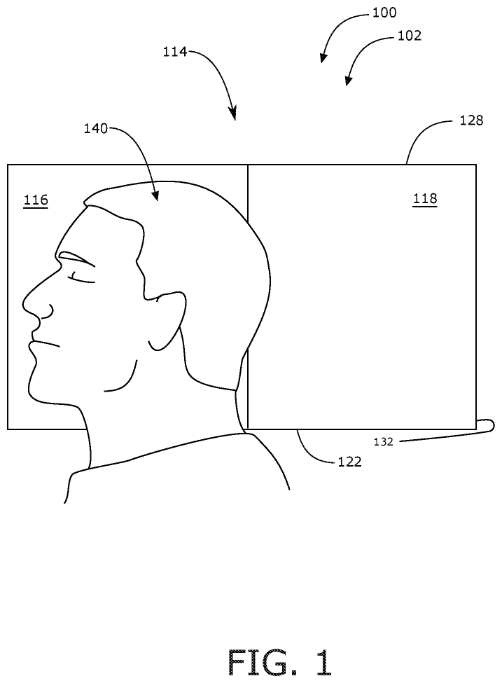

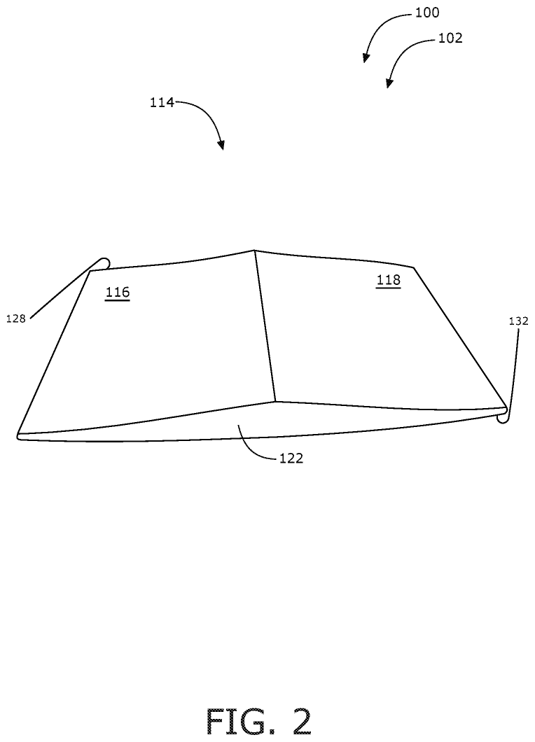

FIGS. 1-4 show anti-snoring system 100 comprising anti-snoring body 102. As shown in FIG. 2, anti-snoring body 102 comprises top side 114, triangle side 1.0 122, triangle side 2.0 128, and bottom side 132. Top side 114 has left surface 116 and right surface 118. Triangle side 1.0 122 lies between left surface 116, right surface 118, and bottom side 132 as does triangle side 2.0 128.

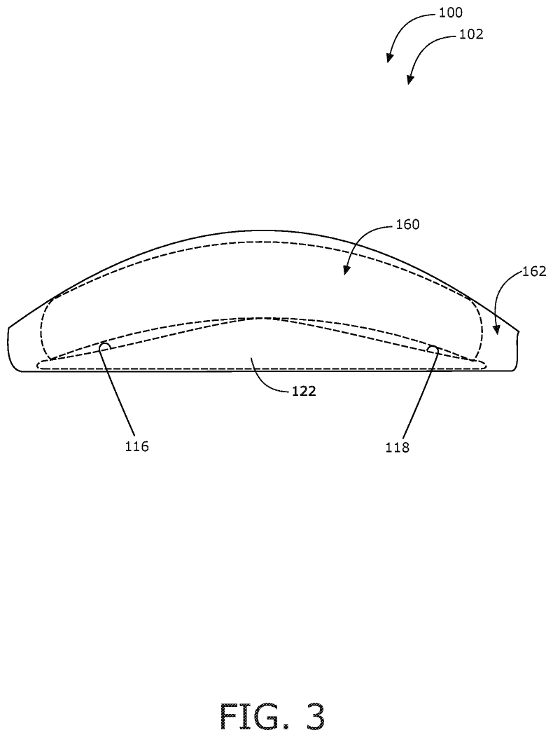

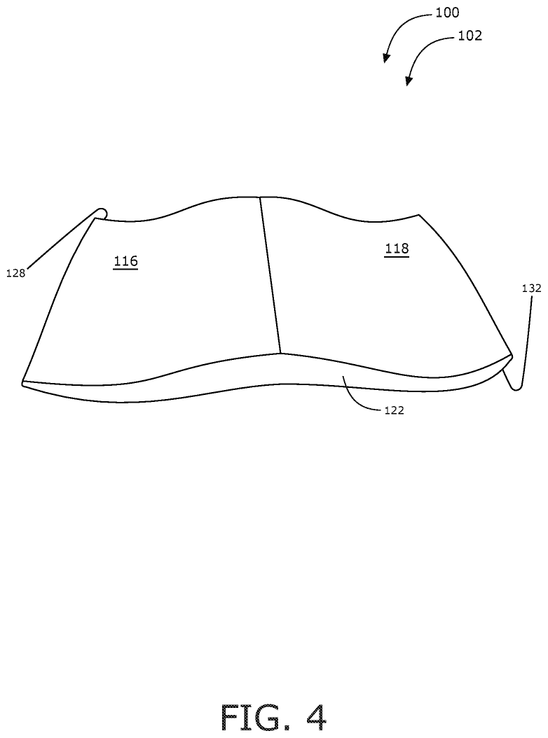

As shown in FIG. 3, anti-snoring system 100 is structured and arranged to be placed beneath pillow 160. It is useful for providing a favorable downward head angle, which may reduce or prevent snoring during sleep. Embodiments may include triangle side 1.0 122 and triangle side 2.0 128, which may be planar, and bottom side 132. In use, bottom side 132 contacts the mattress of a bed. In such embodiments, left surface 116 and right surface 118 are each planar. Alternately, left surface 116 and right surface 118 are each curvilinear (as can be seen in FIG. 4).

Left surface 116 and right surface 118 of top side 114 may be square, rectangular, triangular, or semicircular, depending upon user preferences and manufacturing methods.

Most embodiments of anti-snoring system 100 have a size commensurate with standard sleeping pillows 160. In some versions, anti-snoring system 102 is constructed of a washable material to provide a user with the ability to clean body 102. Body 102 may additionally be constructed of an anti-fungal material to improve hygiene or constructed of a flame retardant material to increase safety.

Materials of manufacture of anti-snoring system 100 may vary, such that anti-snoring system 100 may be constructed of a non-organic, closed-cell foam material for increased longevity and comfort to the user or may be constructed of an organic closed-cell foam material to meet specific user needs or preferences. Additional materials of anti-snoring system 100 may include hypoallergenic materials to provide decreased allergens for the user. Other materials may be used, and different shapes and profiles may apply.

Anti-snoring system 100 may be a kit 440 comprising left and right components of body 102. In these or other versions, kit 440 further comprises at least one pillow 160 and at least one pillowcase 162. Anti-snoring system 100 may be manufactured and provided for sale in a wide variety of sizes and shapes for a wide assortment of applications. Other kit contents or arrangements such as, for example, including more or fewer components, customized parts, different pillow combinations, parts may be sold separately, etc., may be sufficient.

In some embodiments, the device is 18 inches wide (across-the-bed direction), 12 inches long (along-the-bed direction), and 2 inches tall (extending-up-from-the-bed direction). In some embodiments, the device is 14-21, 15-20, 16-19, or 17-18 inches wide. In some embodiments, the device is 10-15, 11-14, or 12-13 inches long. In some embodiments, the device is 1-3 inches tall. In some embodiments, the size affects device function because the size relates to the angle at which the device positions the user.

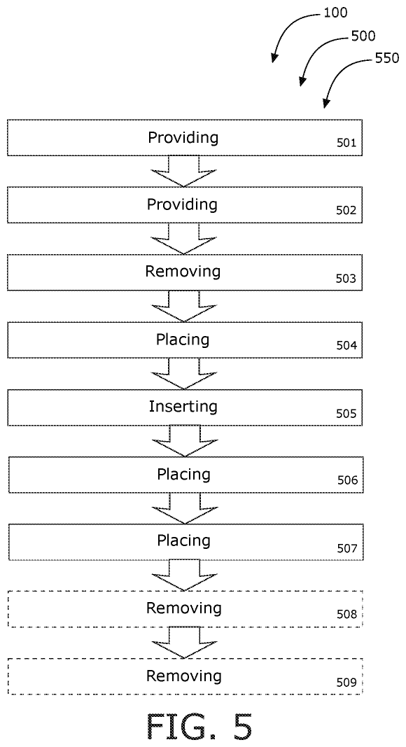

FIG. 5 shows flowchart 550, illustrating method of use 500 for anti-snoring system 100.

As shown, method of use 500 may comprise the steps of step 501, providing anti-snoring body 102; step 502, providing pillow 160 and pillowcase 162; step 503, removing pillow 160 from pillowcase 162; step 504, placing pillow 160 on top of anti-snoring body 102; step 505, inserting anti-snoring body 102 and pillow 160 into pillowcase 162; step 506, placing anti-snoring body 102, pillow 160 and pillowcase 162 upon a user's bed; step 507, placing user's head upon the anti-snoring body 102, pillow 160 and pillowcase 162; step 508, removing anti-snoring body 102, pillow 160 and pillowcase 162; and step 509, removing anti-snoring body from pillowcase 162.

Step 508 and step 509 are each optional and may not occur in all cases. Optional steps of the method of use 500 are illustrated using dotted lines in FIG. 5 to distinguish them from the other steps of the method of use 500.

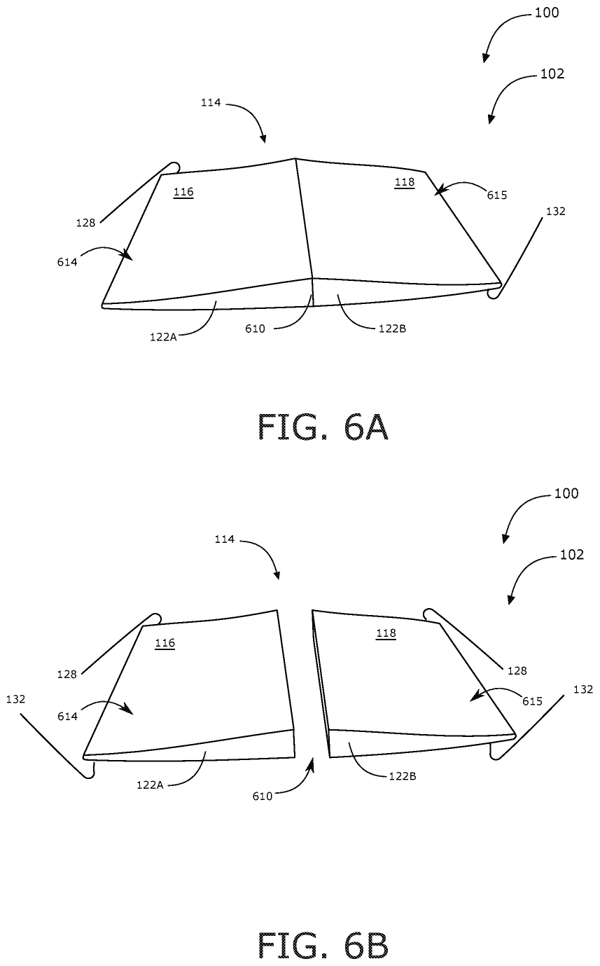

FIGS. 6A and 6B show anti-snoring system 100 comprising anti-snoring body 102. As shown, body 102 comprises top side 114, triangle side 1.0 122A and triangle side 1.1 122B, triangle side 2.0 128, triangle side 2.1 (not shown), and bottom side 132. Top side 114 has left surface 116 and right surface 118. In this version, body 102 comprises a right body 614 and a left body 615. Body sections, right body 614 and left body 615, are similar pieces separated by gap 610. Triangle side 1.0 122 lies between left surface 116, and bottom side 132. Triangle side 1.1 lies between right surface 118 and bottom side 132.

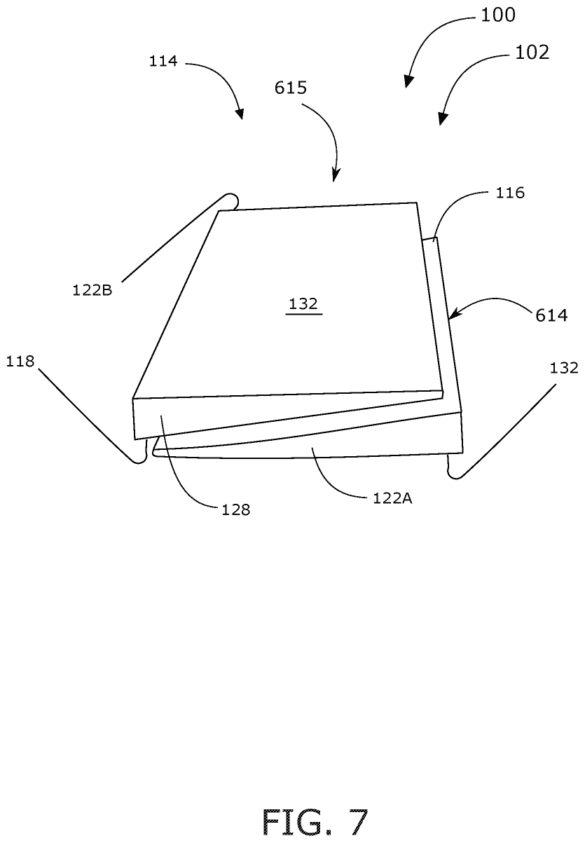

FIG. 7 shows right body 614 and left body 615 of anti-snoring body 102 rearranged into a more compact version, such as is suitable for shipping. As shown, body 102 comprises top side 114, triangle side 1.0 122 A and triangle side 1.1 122B, triangle side 2.0 128, triangle side 2.1 (not shown), and bottom side 132. Top side 114 has left surface 116 and right surface 118. Beginning with the arrangements set out in FIGS. 6A and 6B, left body 615 appears rotated 180° along the axis of gap 610 and 180° horizontally. In this arrangement, left surface 116 and right surface 118 touch each other.

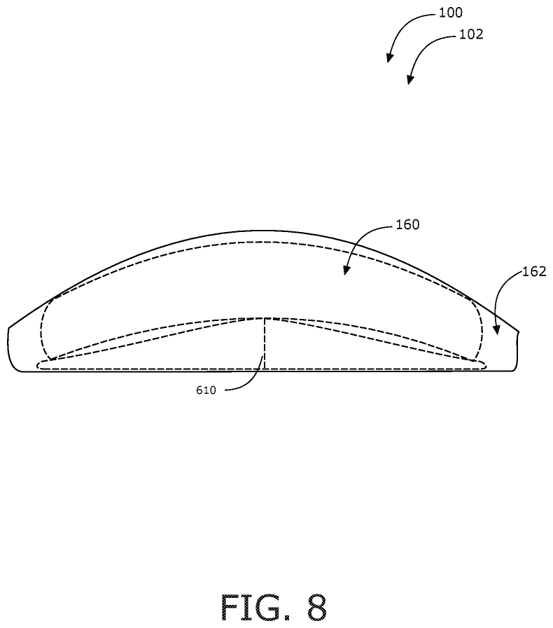

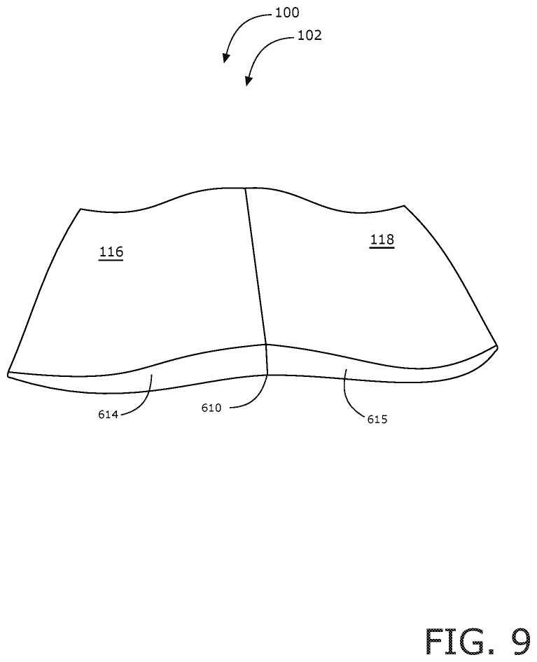

As shown in FIG. 8, anti-snoring body 102 is placed beneath pillow 160. Gap 610 shows how right body 614 and left body 615 are arranged for use. In these versions, as well, body 102 is useful for providing a favorable downward head angle, which may reduce or prevent snoring during sleep. Bottom side 132 contacts the mattress of the bed and raises pillow 160. In such embodiments, left surface 116 and right surface 118 are each planar. Alternatively, left surface 116 and right surface 118 are each curvilinear (as can be seen in FIG. 9). The version shown in both FIGS. 8 and 9 indicates gap 610. Similarly, both figures show right body 614 and a left body 615.

In some embodiments, the anti-snoring system described in this document does not have armholes. In some embodiments, the anti-snoring system places a user's head at 0 to 25°, 0 to 20°, 0 to 15°, 5 to 25°, 5 to 20°, 5 to 15°, 10 to 20°, or 15°. In some embodiments, the anti-snoring system comprises a foam cushion and, in some embodiments, does not contain rigid arcuate components.

Left surface 116 and right surface 118 of top side 114 may be square, rectangular, triangular, or semicircular, depending upon user preferences and manufacturing methods.

Most embodiments of anti-snoring system 100 have a size commensurate with standard sleeping pillows 160. In some versions, anti-snoring system 102 is constructed of a washable material to provide a user with the ability to clean body 102. Body 102 may additionally be constructed of an anti-fungal material to improve hygiene or constructed of a flame retardant material to increase safety.

In some embodiments, the device is 18 inches wide (across-the-bed direction), 12 inches long (along-the-bed direction), and 2 inches tall (extending-up-from-the-bed direction). In some embodiments, the device is 14-21, 15-20, 16-19, or 17-18 inches wide. In some embodiments, the device is 10-15, 11-14, or 12-13 inches long. In some embodiments, the device is 1-3 inches tall.

The steps described in the method of use can occur in many different orders according to user preference. The use of “step of” should not be interpreted as “step for”, in the claims herein and is not intended to invoke the provisions of 35 U.S.C. § 112, ¶ 6. Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within the list mentioned above, elimination or addition of certain steps, including or excluding certain maintenance steps, etc., may be sufficient.

The embodiments of the described invention are exemplary, and numerous modifications, variations, and rearrangements can be readily envisioned to achieve substantially equivalent results, all of which are intended to be embraced within the spirit and scope of the invention. Further, the purpose of the abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is:

1. An anti-snoring system comprising:
a body having an in-use configuration and comprising
a left body with a first triangular side pointing toward the head of a bed, a second triangular side pointing toward the foot of the bed, a bottom surface disposed against the bed, and a top surface facing upward in the in use configuration,
a right body with a first triangular side pointing toward the head of a bed, a second triangular side pointing toward the foot of the bed, a bottom surface disposed against the bed, and a top surface facing upward in the in use configuration,
wherein the system supports a pillow.

2. The system of claim 1, wherein the top surface of the left body and the top surface of the right body meet at an angle.

3. The system of claim 2, wherein the angle is 0 to 25°, 0 to 20°, 0 to 15°, 5 to 25°, 5 to 20°, 5 to 15°, 10 to 20°, or 15°.

4. The system of claim 3, wherein the top surface of the left body and the top surface of the right body each comprise a flat plane.

5. The system of claim 4, wherein, in the in use configuration, the left body connects to the right body through a connection.

6. The system of claim 5, wherein the connection is reversible.

7. The system of claim 6, wherein the body further comprises a stowed configuration in which the left body and the right body stack with angled faces abutting each other.

8. The system of claim 7 further comprising a non-organic, closed-cell foam material.

9. The system of claim 8 further comprising a flame retardant material.

10. The system of claim 9, wherein the body does not contain armholes.

11. The system of claim 7 further comprising an organic, closed-cell foam material.

12. The system of claim 11 further comprising a flame retardant material.

13. The system of claim 12, wherein the body does not contain armholes.

14. The system of claim 7 further comprising a pillow and a pillowcase.

15. An anti-snoring system comprising:
a body having an in-use configuration and comprising
a left body with a first triangular side pointing toward the head of a bed, a second triangular side pointing toward the foot of the bed, a bottom surface disposed against the bed, and a top surface facing upward in the in use configuration and comprising a washable material, a closed-cell foam material or a flame-retardant material,
a right body with a first triangular side pointing toward the head of a bed, a second triangular side pointing toward the foot of the bed, a bottom surface disposed against the bed, and a top surface facing upward in the in use configuration and comprising a washable material, a closed-cell foam material or a flame-retardant material,
wherein
the top surface of the left body and the top surface of the right body meet at a 0 to 25°, 0 to 20°, 0 to 15°, 5 to 25°, 5 to 20°, 5 to 15°, 10 to 20°, or 15° angle,
and
the system supports a pillow.

16. The system of claim 15 further comprising a pillow and a pillowcase.

17. A method comprising:
providing the system of claim 1,
joining the left body and the right body together,
laying the joined left body and right body on a bed,
and
covering the joined left body and right body with a bed pillow.

18. The method of claim 17
wherein the top surface of the left body and the top surface of the right body meet at a 0 to 25°, 0 to 20°, 0 to 15°, 5 to 25°, 5 to 20°, 5 to 15°, 10 to 20°, or 15° angle,
and
further comprising choosing the angle to reduce a user's snoring.

19. The method of claim 18 further comprising repacking the left body and the right body.

20. The method of claim 19 wherein the repacking step includes the steps of
unjoining the left body and right body,
arranging the left body and the right body to form a package shorter than the total length of the joined left body and right body.
